(19) United States
(12) Patent Application Publication
(10) Pub. No.: US 2016/0089392 A1
(43) Pub. Date: Mar. 31, 2016

(54) Whey Protein and Resistant Starch Compositions to Reduce Body Mass Index

(71) Applicant: Fatemeh Malekian, Baton Rouge, LA (US)

(72) Inventor: Fatemeh Malekian, Baton Rouge, LA (US)

(21) Appl. No.: 14/865,158

(22) Filed: Sep. 25, 2015

Related U.S. Application Data
(60) Provisional application No. 62/058,030, filed on Sep. 30, 2014.

(51) Int. Cl. A61K 31/718; A23L 1/22; A61K 9/00; A23L 1/0534; A61K 38/17; A23L 1/305; A23L 1/236

(52) U.S. Cl. CPC A61K 31/718; A23L 1/3056; A23L 1/22; A23L 1/236; A23L 1/0534; A61K 38/1709; A61K 9/0095; A23V 2002/00

(57) ABSTRACT

A nutritional composition comprised of whey protein (WP) and resistant starch (RS) designed to reduce body fat, body fat distribution and as a result lower Body Mass Index (BMI) in humans.

### CROSS-REFERENCES TO RELATED APPLICATIONS

This application claims the benefit of Provisional Application No. 62058030 filed on Sep. 30, 2014.

### Statement Regarding Federally Sponsored Research

This work was supported by the USDA National Institute of Food and Agriculture, [capacity] project #2011-38821-30975

### BACKGROUND

The present invention relates to combination of whey protein (WP) and resistant starch (RS) in the form a nutritional composition.

### BRIEF SUMMARY OF THE INVENTION

There is convincing evidence that a higher WP intake by humans and animals increases satiety and decreases energy intake, body fat, and food intake, compared to diets of lower protein content and can control obesity more than egg albumin, soy protein or casein. In human and animal studies, high protein diets consistently reduce body fat and food intake more than carbohydrate and fat, which lead to a reversal of the metabolic syndrome. Diet composition can have a profound effect on gut gene expression and serum levels of Peptide YY (PYY) and Glucagon like Peotide-1 (GLP-1). Whey protein prevents GLP-1 degradation by inactivating Dipeptidyi peptidase-4 (DPP4) enzyme activity. After ingestion, WP is hydrolyzed into bioactive peptides and amino acids that are sensed by the nervous system at various levels of the gastrointestinal tract and the central nervous system.

The scientific community believes it is the kind of carbohydrate, rather than the amount, in the diet that is important. There are four different types of resistant starches (RSs): RS1 is found in whole grains and legumes and is entrapped in a non-digestible matrix. RS2 is found in foods such as raw potatoes and high amylose cornstarch (high maze resistant starch). The RS3 category includes foods that have undergone “retrogradation,” which occurs when foods containing starches are cooked and then cooled (i.e., potatoes cooled after cooking and puddings). RS4 includes chemically modifying starches with the addition of ester and ether groups and cross-linking amylose strands (breads and cakes). The levels of RS in human diets have been progressively decreased due to modern milling and food preparation methods. Many Americans, especially those of lower socioeconomic status, consume 3-8 g/day of RS in their diets; a level that is lower than intake in medieval Europe (50-100 g/day) or developing countries (30-40 g/day). RS is now available as an ingredient that can be incorporated into breads, cereal and dairy products and other baked goods that are acceptable to the US population. RS has general health benefits for example, several animal studies have shown that RS increases satiety or energy expenditure, decreases plasma cholesterol and triglycerides, increases insulin sensitivity, and produces anticancer effects in humans satiety signals to reduce food intake.

### DETAILED DESCRIPTION OF THE INVENTION

Appetite control of obesity with WP: Decreased body fat as a result of dietary whey protein occurs at several levels of the nervous system between the brain and the gut. There is convincing evidence that a higher WP intake by humans and animals increases satiety (decreases desire to eat), and decreases energy intake, body fat, and food intake, compared to diets of lower protein content and can control obesity more than egg albumin, soy protein or casein. In human and animal studies, high protein diets consistently reduce body fat and food intake more than carbohydrate and fat, which lead to a reversal of the metabolic syndrome. Diet composition can have a profound effect on gut gene expression and serum levels of PYY and GLP-1. Whey protein prevents GLP-1 degradation by inactivating DPP4 enzyme activity. After ingestion, WP is hydrolyzed into bioactive peptides and amino acids that are sensed by the nervous system at various levels of the gastrointestinal tract and the central nervous system. These sensing systems act through satiety signals to reduce food intake.

Reduction of body fat by WP is associated with reduced food intake in rats while, RS is associated with increased energy expenditure without affecting energy intake. Thus, it was proposed that WP and RS may also decrease body fat in humans but by different mechanisms and their effects are additive or synergistic. Therefore, the hypothesis that dietary WP and RS will have the effect of reduced body fat via increased satiety and increased energy expenditure in humans as it does in rats but possibly through different mechanisms was tested.

The nutritional composition comprises whey protein, resistant starch, gum, flavoring and sweetener. Suitable whey protein include whey protein isolate, whey protein concentrate and whey protein hydrolysates. Suitable resistant starch includes RS2 (high maize corn starch) which is available commercially for use in foods. Suitable sweeteners include both natural and artificial sweeteners such as sweet one (acesulfame potassium), aspartame, high-fructose corn syrup, agave nectar, neotame, honey, stevia leaf extract, saccharin, sucralose (Splenda), or sugar alcohols (sorbitol, xylitol, mannitol). Suitable thickening agents include pre-hydrated ticaloid cellulose and derivatives, hemicellulose, pectin, exudate gum, mucilage gum, fructans, alginate, carrageenans, agar, microbial polysaccharides, pullulan, gellan gum, and animal polysaccharides.

The produce the claimed nutritional composition, the calculated amount whey protein, resistant starch, gum, flavoring and sweetener, such as Splenda, were mixed gently with a hand mixer until all the ingredients were well mixed. All the ingredients were in dried and powder form. In another 5 liter mixing bowl all the calculated amount of wheat starch, gum, flavoring and Splenda were mixed for control shakes. Then snack size Ziploc bags were labeled for clarity. Then and 44 grams of mixtures were weight and placed in a bag. The shakes were made for seven days therefore each person received seven bags of the mixture for the whole week. A hand mixer cup, the shake mix (one per day total of seven bags), direction how to make the shakes/smoothies was provided to each participant. They also were given direction on how to make the shake/smoothies with an electric blender.

The participants consumed the shake/smoothie every morning for twenty-four weeks. The participants were advised to use either hand mixer cup or a blender to make the shake or smoothies. It was explained to them to place ½ cup of water in the blender and then add the shake mix and add another ½ cup of water or skim milk/low-fat low sugar almond mike etc. and blend well. They could adjust the thickness of the shake with adjusting the water or milk. They were required to take this shake for breakfast. The participants also were advised to make smoothies if they desired by adding ½ cup to frozen berries or ½ of bananas and ice cubes. Weight and height was measured at the beginning and every Monday for 24 weeks. Body fat distribution was measured using Dual-energy X-ray Absorptiometry (DXA) at the beginning and end of the study. During the intervention the nutrition education intervention/classes for participants (control and treatment group) were provided on how to follow a low fat diet and be physically active for 12 weeks.

The results of proposed research provided knowledge on how WP and RS increased satiety, which led to reduction of food intake. Also it was observed that consumption of smoothies/shakes containing WP and RS in the treatment group decreased food intake compared to the control group. The participants in the treatment group were less hungry and more active and lost weight up to 62 pounds. The combination of reduced food intake and increased energy expenditure/fat burning resulted in reduced body fat in 24 weeks. As a result of the human study being 24 weeks, a significant change in BMI was observed.

The following examples illustrate various non-limiting embodiment of the composition within the present disclosure and are not restrictive of the invention as otherwise described or claimed herein. Compositions may be adjusted for desired taste and consistency.

### EXAMPLES

### Example 1

### Strawberry Shakes:

1. ½ cup whey protein isolate

2. 1 tablespoon resistant Starch

3. 1 tablespoon starch instantized thickener (like guar or xanthan gum)

4. 1 cup water

5. 1 teaspoon of sweetener

6. 1 tablespoon strawberry flavors

7. The formulation was blended until starch or gum reaches desired thickness

Formulation of strawberry flavor shake component percent (by weight) whey protein 11.0%, resistant starch 1.5-3.0%, gum 1.5%, water 83.75%, sweetener 0.75% and strawberry flavors 1.50% total 100%. Compositions may be adjusted for desired taste and consistency.

### Example 2

### Chocolate Shake

1. ½ cup whey protein isolates

2. 1 tablespoon resistant starch

3. 1 tablespoon starch instantized thickener (like guar or xanthan gum)

4. 1 cup of water

5. 1 teaspoon of sweetener

6. 1 tablespoon chocolate flavor

7. The formulation was blended until starch or gum reaches desired thickness

Formulation of chocolate flavor shake component percent (by weight) whey protein 11.0%, resistant starch 1.5-3.0%, gum 1.5%, water 83.75%, sweetener 0.75% and chocolate flavors 1.50% Total 100%. Compositions may be adjusted for desired taste and consistency.

### Example 3

### Vanilla Flavor Shake

1. ½ cup whey protein isolates

2. 1 tablespoon resistant starch

3. 1 tablespoon starch instantized thickener

4. 1 cup of water

5. 1 teaspoon of sugar substitute

6. 1 tablespoon vanilla flavors or extracts

7. The formulation was blended until starch or gum reaches desired thickness.

Formulation of vanilla flavor shake component percent (by weight) whey protein 11.0%, resistant starch 1.5-3.0%, gum 1.5%, water 83.75%, sweetener 0.75% and chocolate flavors 1.50% Total 100%. Compositions may be adjusted for desired taste and consistency.

### Example 4

Smoothie with Frozen Fruits

1. ½ cup whey protein isolates

2. 1 tablespoon resistant starch

3. 1 tablespoon starch instantized thickener (like guar or xanthan gum)

4. 1 cup of water

5. 1 teaspoon of sugar substitute

6. 1 tablespoon vanilla flavors or extracts

7. ½ cup of frozen berries

8. The formulation was blended until mix reaches desired thickness.

Formulation of vanilla flavor smoothie component percent (by weight) whey protein 9.0%, resistant starch 1.25-2.5%, gum 1.25%, water 64.50%, sweetener 0.75% and chocolate flavors 1.25%, frozen berries 22.0% Total 100%. Compositions may be adjusted for desired taste and consistency.

### Example 5

Smoothie with Fresh Fruits

1. ½ cup whey protein isolates

2. 1 tablespoon resistant starch

3. 1 tablespoon starch instantized thickener (like guar or xanthan gum)

4. 1 cup of water

5. 1 teaspoon of sugar substitute

6. 1 tablespoon vanilla flavors or extracts

8. ½ cup of fresh fruits

9. ½ cup of ice (crushed or cubes)

10. The formulation was blended until mix reaches desired thickness.

Formulation of vanilla flavor smoothie component percent (by weight) whey protein 7.0%, resistant starch 0.9-1.8%, gum 0.9%, water 51.85%, sweetener 0.45% and vanilla flavors 0.9%, fresh fruits 12.5%, crushed ice 25.5% Total 100%. Compositions may be adjusted for desired taste and consistency.

I claim:

1. A nutritional composition comprising:
a. Whey protein,
b. Resistant starch,
c. Flavoring,
d. Sweetener,
e. A means of thickening, and
f. A consumable liquid.

2. The nutritional composition of claim 1, wherein:
a. Said whey protein comprises up to 11% weight/volume of the nutritional composition,
b. Said resistant starch comprises between about 1.5% to 3.0% weight/volume of the nutritional composition,
c. Said flavoring comprises up to about 1.5% weight/volume of the nutritional composition,
d. Said sweetener comprises up to about 0.75% weight/volume of the nutritional composition,
e. Said means of thickening comprises up to about 1.5% weight/volume of the nutritional composition, and
f. Said consumable liquid comprises about 84% weight/volume of the nutritional composition.

3. The nutritional composition of claim 2, wherein said resistant starch comprises RS2.

4. The nutritional composition of claim 3, wherein said whey protein comprises whey protein isolate.

5. The nutritional composition of claim 4, wherein said means of thickening comprises pre-hydrated ticaloid.

6. The nutritional composition of claim 5, wherein:
a. Said sweetener comprises an artificial sweetener, and
b. Said consumable liquid is water.

7. The nutritional composition of claim 1, further comprising fruit and ice.

8. The nutritional composition of claim 7, wherein:
a. Said whey protein comprises up to about 7.0% weight/volume of the nutritional composition,
b. Said resistant starch comprises between about 0.9% to 1.8% weight/volume of the nutritional composition,
c. Said flavoring comprises up to about 0.9% weight/volume of the nutritional composition,
d. Said sweetener comprises up to about 0.45% weight/volume of the nutritional composition,
e. Said means of thickening comprises up to about 0.9% weight/volume of the nutritional composition,
f. Said consumable liquid comprises about 51.85% weight/volume of the nutritional composition,
g. Said fruit comprises about 12.5% weight/volume of the nutritional composition, and
h. Said ice comprises about 25.5% weight/volume of the nutritional composition.

9. The nutritional composition of claim 8, wherein said resistant starch comprises RS2.

10. The nutritional composition of claim 9, wherein said whey protein comprises whey protein isolate.

11. The nutritional composition of claim 10, wherein said means of thickening comprises pre-hydrated ticaloid.

12. The nutritional composition of claim 12, wherein:
a. Said sweetener is an artificial sweetener; and
b. Said consumable liquid is water.

13. A nutritional composition comprising:
a. Whey protein isolate up to about 11.0% weight/volume of the nutritional composition,
b. Resistant starch between about 1.5% to 3.0% weight/volume of the nutritional composition,
c. Flavoring up to about 1.5% weight/volume of the nutritional composition,
d. Sweetener up to about 0.75% weight/volume of the nutritional composition,
e. A means of thickening up to about 1.5% weight/volume of the nutritional composition, and
f. A consumable liquid comprises about 83.75% weight/volume of the nutritional composition.

14. The nutritional composition of claim 13, wherein:
a. Said means of sweetener is an artificial sweetener,
b. Said means of thickening comprises pre-hydrated ticaloid, and
c. Said consumable liquid is water.

15. The nutritional composition of claim 14, wherein said resistant starch comprises RS2.

16. A method of producing a nutritional composition comprising:
a. Providing whey protein up to about 11.0% weight/volume of the nutritional composition,
b. Providing resistant starch between about 1.5% to 3.0% weight/volume of the nutritional composition,
c. Providing flavoring up to about 1.5% weight/volume of the nutritional composition,
d. Providing a sweetener up to about 0.75% weight/volume of the nutritional composition,
e. Providing a means of thickening up to about 1.5% weight/volume of the nutritional composition,
f. Providing a consumable liquid comprises about 83.75% weight/volume of the nutritional composition, and
g. Blending the nutritional composition for over sixty seconds.

17. The method of claim 16, wherein:
a. Said whey protein comprises whey protein isolate,
b. Said resistant starch is RS2,
c. Said means of thickening comprises pre-hydrated ticaloid,
d. Said sweetener comprises an artificial sweetener, and
e. Said consumable liquid is water.
